(19) United States
(12) Patent Application Publication
(10) Pub. No.: US 2010/0113495 A1
(43) Pub. Date: May 6, 2010

(54) PHARMACEUTICAL COMPOSITIONS COMPRISING AN OPIOID RECEPTOR ANTAGONIST AND METHODS OF USING SAME

(76) Inventors: Daniel Wermeling, Lexington, KY (US); Edwin A. Cohen, Mahwah, NJ (US); Anita C. Rudy, Nicholasville, KY (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/477,390

(22) Filed: Jun. 3, 2009

Related U.S. Application Data
(63) Continuation of application No. 11/673,874, filed on Feb. 12, 2007, now abandoned.
(60) Provisional application No. 60/771,995, filed on Feb. 10, 2006.

(51) Int. Cl. A61K 31/485; A61P 25/32

(52) U.S. Cl. 514/282

(57) ABSTRACT

The present invention features compositions for intranasal administration comprising an opioid receptor antagonist. The invention also features methods of using such compositions in the treatment of various diseases and disorders, such as the treatment of alcoholism. In certain embodiments, the opioid receptor antagonist is naltrexone or a pharmaceutically acceptable salt thereof.

### RELATED APPLICATIONS

This application claims the benefit of priority to U.S. Provisional Patent Application Ser. No. 60/,771,995, filed Feb. 10, 2006, the contents of which are hereby incorporated by reference.

### FIELD OF THE INVENTION

The present invention relates to pharmaceutical compositions comprising an opioid receptor antagonist and to methods of using such compositions to treat and/or prevent various diseases and disorders, such as alcoholism and/or alcohol cravings.

### BACKGROUND OF THE INVENTION

Alcoholism is a disease impacting more than an estimated 10-15 million people in the United States alone and some 70 million or more worldwide. Alcohol, globally, contributes to more than 1.5 million annual deaths. Overall, there are causal relationships between alcohol consumption and more than 60 types of disease and injury including traffic fatalities. Alcohol consumption is the leading risk factor for disease burden in low mortality developing countries and the third largest risk factor in developed countries. In all, it is estimated that the global annual cost of treating alcohol abuse is more than $250 billion.

Patients suffering from alcoholism typically require a comprehensive program of medical, pharmacologic and behavioral intervention. Pharmacologic therapy for alcoholism typically has employed one or more of several oral therapies designed to decrease ethanol cravings. Such oral therapies include disulfuram, naltrexone and acamprosate, among others. Unfortunately, these interventions are not very effective with many patients still experiencing craving symptoms as part of their daily experience, or relapsing altogether.

Naltrexone was initially introduced for alcohol dependence in the United States around 1994 as an oral tablet. Results of several clinical trials considered in aggregate suggest that naltrexone added to substance abuse treatment or counseling reduces heavy drinking among patients who take the drug as prescribed. Unfortunately, however, overall efficacy of oral naltrexone is not always significantly better than placebo in clinical studies. One of the main factors influencing the effectiveness of naltrexone is poor patient compliance with therapy. Many patients prescribed oral naltrexone do not take all of their medication because, inter alia, they experience side effects such as fatigue and nausea that may be due to the poor oral bioavailability of the drug and/or extensive first pass metabolism and metabolite (e.g. naltrexol) formation. Oral naltrexone, for example, exhibits oral bioavailability of only about 10%-25% for a 50 mg oral dose. This means that excessive oral doses of naltrexone must be administered in order to provide a therapeutically effective amount of drug to a patient.

If improved treatments and/or preventatives for alcoholism and related disorders could be developed, a significant advance in the art would result.

### SUMMARY OF THE INVENTION

In various embodiments, the present invention provides intranasal compositions and methods for using the same in the treatment and/or prevention of various diseases and disorders, such as alcoholism and ethanol craving.

Alcoholism (also referred to as alcohol dependence) generally refers to a compulsion to seek and consume alcohol, a loss of control over consumption after beginning a drinking session, and/or a strong likelihood of relapse during or after withdrawal from alcohol consumption. These manifestations may be accompanied by a conscious desire or urge to consume alcohol (i.e., craving). Craving can occur spontaneously, or it can be elicited by internal or external stimuli, known as cues. Internal cues may include emotional states (e.g., anxiety) or symptoms of acute alcohol withdrawal. External cues may include exposure to alcohol-related environments or objects, e.g., bottles of alcoholic beverages, advertisements, etc.

In one embodiment, the present invention provides a pharmaceutical composition for intranasal administration to a mammal comprising a therapeutically effective amount of an opioid receptor antagonist, a liquid nasal carrier, and optionally one or more pharmaceutically acceptable excipients.

The related terms “therapeutically effective amount,” “prophylactically effective amount,” or “effective amount” as used herein refer to an amount of drug or agent that is sufficient to elicit the required or desired therapeutic and/or prophylactic response, as the particular treatment context may require.

In another embodiment, the present invention provides a method of treating a mammal comprising intranasally administering to the mammal an effective amount of a composition as described herein. In a related embodiment, the mammal suffers from alcoholism or ethanol cravings.

In another embodiment, the present invention provides an intranasal unit-dose delivery device comprising one or more sealed vessels or containers comprising a sterilized, pharmaceutical composition as described herein. In a related embodiment, upon positioning the device a fixed distance away from a detection laser beam, actuating the device to produce a spray plume perpendicular to the laser beam, and detecting droplet size distribution of the spray plume with the laser beam, the spray plume has defined droplet size dispersion characteristics.

In another embodiment, upon positioning the device a fixed distance away from an impaction plate, actuating the device to produce a spray pattern onto the impaction plate, and measuring the diameter of the spray pattern, the spray pattern has a defined maximum diameter, minimum diameter and/or span.

These and other embodiments of the present invention are described in more detail herein below.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

### Opioid Receptor Antagonists

Compositions of the invention comprise at least one pharmaceutically acceptable opioid receptor antagonist. The term “opioid receptor antagonist” as used herein includes any substance that selectively blocks an opioid receptor of any type (e.g., μ, δ, K, etc.) or subtype (e.g., μ₁/μ₂). Suitable opioid receptor antagonists for use in the present invention include, but are not limited to, naltrexone, nalmefene, naloxone, naloxonazine, nor-binaltorphimine, naltrindole or combinations thereof.

The opioid receptor antagonist may be in free form or in pharmaceutically acceptable salt or complex form. “Pharmaceutically acceptable salts,” or “salts,” include the salt of an opioid prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, betahydroxybutyric, galactaric and galacturonic acids.

In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. Non-limiting examples of pharmaceutically acceptable salts of opioids include those salt-forming acids and bases that do not substantially increase the toxicity of the compound. Non-limiting examples of suitable salts include salts of alkali metals such as magnesium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like. In certain embodiments, the opioid receptor antagonist is naltrexone hydrochloride.

In other embodiments, an acid addition salt is reconverted to the free base by treatment with a suitable base. In a further embodiment, suitable acid addition salts of the opioid are halide salts, which are prepared using hydrochloric or hydrobromic acids. In still other embodiments, the basic salts are alkali metal salts, e.g., sodium salt.

Compositions of the invention can comprise one or more opioid receptor antagonists in any suitable amount. In one embodiment, a composition of the invention comprises an opioid receptor antagonist in an amount of about 1 μg to about 100 mg, about 1 μg to about 80 mg, about 1 μg to about 50 mg, or about 1 μg to about 40 mg. In other embodiments, a composition of the invention comprises an opioid receptor antagonist in an amount of about 1 mg to about 100 mg, about 1 mg to about 80 mg, about 1 mg to about 50 mg, or about 1 mg to about 40 mg. Compositions of the invention typically comprise one or more opioid receptor antagonists in a concentration of about 0.01 mg/mL to about 10 mg/mL, about 0.01 mg/mL to about 1.0 mg/mL, about 0.1 mg/mL to about 300 mg/mL, about 0.5 mg/mL to about 250 mg/mL, about 0.75 mg/mL to about 200 mg/mL, or about 1 mg/mL to about 100 mg/mL.

In one embodiment, the opioid receptor antagonist is naltrexone or a pharmaceutically acceptable salt thereof, and is present in the composition in a concentration of about 10 mg/mL to about 200 mg/mL, about 10 mg/mL to about 100 mg/mL, about 30 mg/mL to about 150 mg/mL, about 40 mg/mL to about 120 mg/mL, about 50 mg/mL to about 110 mg/mL, about 60 mg/mL to about 100 mg/mL, or for example about 80 mg/mL.

### Liquid Nasal Carrier

Compositions of the present invention comprise a liquid carrier. As used herein, the phrases “liquid carrier” or “liquid nasal carrier” refer to a liquid vehicle (e.g., solution, emulsion, or suspension) designed for delivery of a drug to the nasal mucosa of a subject. The liquid nasal carrier can include one or more excipients such as diluents, solvents and/or co-solvents suitable for application to the nasal mucosa. Suitable diluents include aqueous or non-aqueous diluents or combination thereof. Examples of aqueous diluents include, but are not limited to, saline, water, water for injection (WFI), dextrose or combinations thereof.

In one embodiment, the liquid nasal carrier comprises a solvent such as a water miscible solvent. Non-limiting examples of suitable solvents include propylene glycol, alcohol, glycerol, isopropyl alcohol and polyethylene glycol.

Any desired aqueous and/or non-aqueous diluents, solvents or co-solvents can be added in various concentrations and combinations to form compositions of the invention. The liquid nasal carrier can be present in any suitable amount, for example about 10% to about 99%, about 20% to about 98%, about 30% to about 97%, by weight-in-volume (w/v) of the composition. In another embodiment, the liquid nasal carrier can be added to the other components of the composition in an amount sufficient to q.s. the composition to a desired final volume.

In one embodiment, at least a portion of, at least a therapeutically effective portion of, at least about 20% (w/v), at least about 50% (w/v), at least about 75% (w/v), at least about 90% (w/v), or substantially all of the opioid receptor antagonist is in dissolved or solubilized form in the liquid carrier.

### Pharmaceutical Excipients

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose formulation of the composition.

Illustrative excipients include antioxidants, surfactants, adhesives, agents to adjust the pH and osmolarity, preservatives, antioxidants, thickening agents, sweetening agents, flavoring agents, taste masking agents, colorants, buffering agents, and penetration enhancers. Generally speaking, a given excipient, if present, will be present in an amount of about 0.001% to about 20% (w/v), about 0.01% (w/v) to about 10% (w/v), about 0.02% (w/v) to about 5% (w/v), or about 0.3% (w/v) to about 2.5% (w/v).

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% (w/v) to about 2.5% (w/v), for example about 0.01% (w/v), about 0.05% (w/v), about 0.1% (w/v), about 0.5% (w/v), about 1% (w/v), about 1.5% (w/v), about 1.75% (w/v), about 2% (w/v), about 2.25% (w/v), or about 2.5% (w/v).

In various embodiments, compositions of the invention comprise a preservative. Ideally, the optional preservative will be present in quantities sufficient to preserve the composition, but in quantities low enough that they do not cause irritation of the nasal mucosa. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% (w/v) to about 0.5% (w/v) or about 0.01% (w/v) to about 2.5% (w/v).

In other embodiments, compositions of the invention are preservative-free. As used herein, the term “preservative-free” includes compositions that do not contain any preservative. Thus, in various embodiments, the composition does not contain, for example, benzalkonium chloride, methyl, ethyl, propyl or butylparaben, benzyl alcohol, phenylethyl alcohol, or benzethonium.

In one embodiment, compositions of the invention optionally comprise a buffering agent. The optional buffering agent, if present, is present in a composition of the invention in an amount that does not irritate the nasal mucosa. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline earth metal buffering agent, an aluminum buffering agent, a calcium buffering agent, a sodium buffering agent, or a magnesium buffering agent. Other suitable classes of buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (Based in part upon the list provided in The Merck Index, Merck & Co. Rahway, N.J. (2001)). Further more, combinations or mixtures of any two or more of the above mentioned buffering agents can be used in the pharmaceutical compositions described herein. One or more buffering agents, if desired, are present in compositions of the invention in an amount of about 0.01% (w/v) to about 5% (w/v) or about 0.01% (w/v) to about 3% (w/v).

In one embodiment, compositions of the invention optionally comprise one or more surfactants. Optional surfactants are typically present in a composition of the invention in an amount of about 0.1 mg/mL to about 10 mg/mL, about 0.5 mg/mL to 5 mg/mL or about 1 mg/mL.

In various embodiments, compositions the invention may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% (w/v) to about 10% (w/v), or about 0.1% (w/v) to about 5% (w/v).

In various embodiments, compositions of the invention comprise one or more sweeteners and/or flavoring agents. Suitable sweeteners and/or flavoring agents include any agent that sweetens or provides flavor to a pharmaceutical composition. The sweetener or flavoring agent will help mask any bitter or bad taste that may occur if the pharmaceutical composition drips back into the mouth after intranasal administration. By addition of a sweetener or flavoring agent to the intranasal composition, a barrier that a patient may have to taking the intranasal composition because of unpleasant taste can be reduced. Optional sweetening agents and/or flavoring agents are typically present in a composition of the invention in an amount of about 0.1 mg/mL to about 10 mg/mL, about 0.5 mg/mL to 5 mg/ml or about 1 mg/mL.

Illustrative sweeteners or flavoring agents include, without limitation, acacia syrup, anethole, anise oil, aromatic elixir, benzaldehyde, benzaldehyde elixir, cyclodextrins, caraway, caraway oil, cardamom oil, cardamom seed, cardamom spirit compound, cardamom tincture compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, isoalcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit compound, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof.

Illustrative taste masking agents include, but are not limited to, cyclodextrins, cyclodextrins emulsions, cyclodextrins particles, cyclodextrins complexes, or combinations thereof.

The foregoing excipients can have multiple roles as is known in the art. For example, some flavoring agents can serve as sweeteners as well as a flavoring agent. Therefore, classification of excipients above is not to be construed as limiting in any manner.

Pharmaceutical compositions as disclosed herein are not limited to any particular pH. In one embodiment, pH of a composition of the invention ranges from about 3 to about 7, about 3 to about 6, or about 4 to about 6, for example about 5. If adjustment of pH is needed, it can be achieved by the addition of an appropriate acid, such as hydrochloric acid, or base, such as for example, sodium hydroxide.

Pharmaceutical compositions of the invention can be prepared in any suitable manner. In one embodiment, the compositions are prepared by mixing an opioid receptor antagonist (e.g. naltrexone) with a liquid nasal carrier and one or more optional excipients at room temperature under aseptic conditions. In other embodiments, the mixture can be prepared under non-aseptic conditions and then sterile filtered, autoclaved or otherwise sterilized and packaged in a delivery device. It will be understood by those of ordinary skill in the art that the order of mixing is not critical, and the present invention includes without limitation mixing of compositions of the invention in any order.

### Stability

In one embodiment, a composition of the invention comprises at least about 85%, at least about 87%, at least about 90%, at least about 92%, at least about 95%, at least about 97%, or at least about 99% of the original opioid receptor antagonist after storage at 40° C. and 75% relative humidity for a period of at least about 1 week, at least about 2 weeks, at least about 3 weeks, at least about 4 weeks, at least about 6 weeks, at least about 8 weeks, at least about 10 weeks, at least about 15 weeks, at least about 20 weeks, at least about 25 weeks, at least about 30 weeks, at least about 35 weeks, at least about 40 weeks, at least about 45 weeks, or at least about 50 weeks.

### Methods of Treatment

Compositions as described herein can be used in accordance with various methods of treatment and/or prevention of diseases and disorders. Any of the various compositions described herein can be used in any of the methods described.

In one embodiment, compositions of the invention are useful in the treatment and/or prevention of alcoholism and other diseases and disorders. In certain embodiments, compositions of the invention are sprayed or discharged into the nasal cavity of a subject. In certain embodiments, each spray or discharge will typically provide a volume of about 25 μL to about 250 μL, about 50 μL to about 200 μL, about 75 μL to about 175 μL, for example, about 100 μL of a composition as described herein. In certain embodiments, each spray or discharge will typically provide a volume of about 1 μL to about 1000 μL, about 25 μL to about 500 μL, about 50 μL to about 250 μL, or about 50 μL to about 150 μL of a composition described herein.

In certain embodiments, intranasal administration of a composition of the invention provides a dose in the range of about 0.5 mg to about 15 mg of an opioid receptor antagonist, about 1 mg to about 10 mg of an opioid receptor antagonist, about 2 mg to about 8 mg of an opioid receptor antagonist, about 4 mg to about 6 mg of an opioid receptor antagonist, or about 5 mg to about 8 mg of an opioid receptor antagonist to the nasal cavity of a subject. In certain embodiments, the opiod receptor antagonist is naltrexone or a salt thereof.

In another embodiment, a composition of the invention, upon intranasal administration to a subject, provides absolute bioavailability of the opioid receptor antagonist (e.g. naltrexone) of at least about 40%, at least about 50%, at least about 60%, at least about 70%, or at least about 80%, for example about 40% to about 99%, about 50% to about 90%, or about 60% to about 80%, where absolute bioavailability is compared to intravenous dose administration. In certain embodiments, intranasal administration of an opioid receptor antagonist to a subject provides absolute bioavailability of the opioid receptor antagonist in the range of about 20% to about 80%, about 30% to about 70%, about 40% to about 70%, about 50% to about 70%, about 60% to about 70%, or about 50% to about 60%, where absolute bioavailability is compared to intravenous dose administration.

In another embodiment, a composition of the invention is intranasally administered to a subject who is concurrently prescribed to take and/or is taking oral or injectable naltrexone (or salt thereof) or other opioid receptor antagonist. In a related embodiment, a subject currently taking oral or injectable naltrexone (or salt thereof) to treat or prevent alcoholism will be administered intranasal naltrexone (or salt thereof) when he or she experiences a craving for alcohol or senses that a relapse to alcohol dependence is likely imminent.

In another embodiment, a composition of the invention is intranasally administered to a subject that has relapsed to consumption of alcohol after and/or while taking one or more other medications to treat or prevent (directly or indirectly) alcohol consumption.

In another embodiment, a composition of the invention is intranasally administered to a subject in need of acute extinguishment of alcohol craving symptoms. In another embodiment, the present invention provides a method for reducing the number of drinks per unit time (e.g. per hour, per day or per week) that a subject consumes, the method comprising intranasally administering to a subject in need thereof a composition as described herein.

In another embodiment, a composition of the invention is administered in combination (as defined herein below) with one or more of acamprosate, tiapride, ondensetron, sertraline, topirimate, aripiprazole and/or disulfiram.

In another embodiment, the present invention provides a method for reducing naltrexol formation upon administration of naltrexone to a subject (by comparison with the amount of naltrexol formed after oral administration of an equivalent dose of naltrexone), the method comprising intranasally administering naltrexone to a subject in need of such treatment. In one embodiment, upon intranasal administration of a naltrexone composition of the invention, the weight ratio of plasma naltrexol (a formed metabolite) to plasma naltrexone is not more than about 9:1, about 8:1, about 7:1, about 6:1. about 5:1, about 4:1, about 3:1, about 2:1, about 1:1, about 1:2, about 1:3, about 1:4, about 1:5, about 1:6, about 1:7, about 1:8, about 1:9 or about 1:10.

In another embodiment, the present invention provides a method for treating and/or preventing alcoholism in a subject in need thereof comprising administering to a subject in need thereof a combination of one or more of oral, injectable and/or intranasal compositions comprising an opioid receptor antagonist (e.g. naltrexone or salt thereof). The term “combination” in the context of combination administration of different, therapeutic agents, compositions or different dosage forms of the same compound means that the agents, compounds, or different dosage forms thereof (e.g. oral, injectable and/or intranasal) are administered substantially simultaneously, concurrently, in a manner to result in overlap of, or alternation of therapeutic activity, or sequentially as needed or desired over any given period of at least about 1 day, at least about 5 days, at least about 10 days, at least about 20 days, at least about 30 days, at least about 60 days, at least about 90 days, at least about 180 days or at least about 360 days. For example, combination therapy can include administration of oral naltrexone (or salt thereof) substantially daily over a period of at least about 5 days, at least about 10 days, at least about 20 days, at least about 30 days, at least about 60 days, at least about 90 days, at least about 180 days or at least about 360 days or longer, and intranasal administration of naltrexone (or salt thereof) as desired or needed during that period. The intranasal naltrexone administration can occur as often as desired throughout the treatment period, for example once per day, once per week, or once per month.

In a related embodiment, the present invention provides a method for treating and/or preventing alcoholism in a subject in need thereof comprising administering to a subject a combination of oral naltrexone (or salt thereof) and intranasal opioid receptor antagonist (e.g. naltrexone or salt thereof). For example, the subject can be administered about 10 mg to about 100 mg, about 20 mg to about 80 mg, or about 30 mg to about 60 mg, for example about 50 mg of oral naltrexone (or salt thereof) per day, and can be administered an intranasal naltrexone (or salt thereof) composition as described herein about 0, about 1, about 2, about 3, about 4 or about 5 times per day, or for example as frequently as is needed to help prevent or reduce relapse or to prevent or reduce the impact of perceived or non-perceived alcohol cravings. The intranasal administration can also occur about once per day, once per week, or once per month during the oral treatment period.

In another related embodiment, the present invention provides a method for treating and/or preventing alcoholism in a subject in need thereof comprising administering to a subject a combination of injectable naltrexone (or salt thereof) and intranasal opioid receptor antagonist (e.g. naltrexone or salt thereof). For example, the subject can be administered about 10 mg to about 500 mg, 20 mg to about 450 mg, or about 30 mg to about 400 mg of naltrexone about once per day to about once per month, for example about once per day, about once per 5 days, about once per 10 days, about once per 20 days, or about once per 30 days; the subject is also administered an intranasal naltrexone (or salt thereof) composition as described herein about 0, about 1, about 2, about 3, about 4 or about 5 times per day, or for example as frequently as is needed to help prevent or reduce relapse or to prevent or reduce the impact of perceived or non-perceived alcohol cravings. The intranasal administration can also occur about once per day, once per week, or once per month during the injectable treatment period.

In another embodiment, where the drug being delivered is naltrexone (or salt thereof), upon intranasal administration of a composition of the invention to a subject, the subject exhibits one or more of: a Tₘₐₓ naltrexone plasma concentration of less than about 0.75 hr (for example about 5 minutes to about 45 minutes after administration, or about 10 minutes to about 25 minutes); a Cₘₐₓ naltrexone plasma concentration of at least about 2 ng/mL, for example about 2 ng/mL to about 30 ng/mL, about 5 ng/mL to about 20 ng/mL, or about 10 ng/mL to about 20 ng/mL; and/or an AUC naltrexone plasma concentration of at least about 5 ng*hr/mL, for example about 5 to about 40 ng*hr/mL, or about 10 to about 30 ng*hr/mL. In a related embodiment, the above PK parameters result after administration of a composition in an amount sufficient to provide the subject with about 10 mg to about 40 mg of naltrexone moiety.

### Delivery Device

Compositions of the present invention can be administered using any suitable intranasal delivery device. In one embodiment, the delivery device is a unit-dose delivery device. Delivery devices comprising any of the pharmaceutical compositions of various embodiments disclosed herein comprise further embodiments of the invention. Non-limiting examples of suitable intranasal delivery devices, or components thereof, are disclosed in the following U.S. Patents and U.S. Patent Publications, each of which are hereby incorporated by reference herein in their entirety: U.S. Pat. No. 4,946,069; U.S. Pat. No. 5,307,953; U.S. Pat. No. 5,368,201; U.S. Pat. No. 5,395,032; U.S. Pat. No. 5,427,280; U.S. Pat. No. 5,482,193; U.S. Pat. No. 5,584,417; U.S. Pat. No. 5,813,570; U.S. Pat. No. 5,893,484; U.S. Pat. No. 5,944,222; U.S. Pat. No. 5,964,417; U.S. Pat. No. 5,967,369; U.S. Pat. No. 6,062,433; U.S. Pat. No. 6,257,454; U.S. Pat. No. 6,626,379; U.S. Pat. No. 6,321,942; U.S. Pat. No. 6,367,473; and U.S. Pat. No. 6,948,492.

The delivery device can be filled with single or multidose amounts of opioids. In one embodiment, the vessel or container holding the pharmaceutical composition and its sealing means are sterilizable. In one such embodiment, the parts of the device that are in contact with the pharmaceutical composition can be constructed and assembled in a configuration so as to allow for sterilization. Devices with one or more unit-dose(s) can be sterilized either before or after filling and/or packaging, employing methods and technology that are well known in the art. Individual devices can be packaged, sterilized and shipped; alternatively, entire shipping and storage packages can be sterilized at once, and the devices removed individually for dispensing, without affecting the sterility of the remaining units.

In one embodiment, the volume of liquid contained in each vessel of a delivery device is about 0.025 mL to about 2 mL, about 0.25 mL to 1 mL, or about 0.05 mL to about 0.15 mL.

In another embodiment, a composition of the invention, upon being discharged from an intranasal spray device at a spray distance of 1 cm from a detection laser, for example at a discharge volume of about 100 μL per spray, exhibits a droplet size distribution having a mean Dv10 of about 5 to about 50 μm, about 7.5 to about 40 μm, or about 10 to about 35 μm; a mean Dv50 of about 15 to about 80 μm, about 20 to about 70 μm, or about 30 to about 60 μm; and/or a mean Dv90 of about 40 to about 130 μm, about 50 to about 120 μm, or about 60 to about 100 μm. In another embodiment, the spray has a mean span [(Dv90-Dv10/Dv50)] of about 1 to about 5, about 1.25 to about 4, or about 1.5 to about 3.

In a related embodiment, upon positioning the device 1 cm away from an impaction plate, actuating the device to produce a spray pattern onto the impaction plate, and measuring the diameter of the spray pattern the spray pattern has a maximum diameter (Dₘₐₓ) of about 1 to about 4 cm, about 2 to about 3 cm or about 2.2 to about 2.5 cm, for example about 2.3 cm. In another related embodiment, the spray has a minimum diameter (Dₘᵢₙ) of about 1 to about 3 cm, about 1.5 to about 2.8 cm or about 1.8 to about 2.3 cm, for example about 2.1 cm.

### Exemplary Compositions

One aspect of the invention features an intranasally deliverable pharmaceutical composition comprising a therapeutically effective amount of an opioid receptor antagonist or salt thereof and a liquid carrier. In certain embodiments, the opioid receptor antagonist comprises naltrexone or salt thereof. In certain embodiments, the naltrexone or salt thereof is present in dissolved or solubilized form in the liquid carrier at a concentration of about 10 mg/mL to about 100 mg/mL. In certain embodiments, the liquid carrier further comprises at least one water miscible solvent. In certain embodiments, the water miscible solvent comprises propylene glycol, alcohol, glycerol, isopropyl alcohol or polyethylene glycol, or mixtures thereof. In certain embodiments, upon intranasal administration of the composition to a human subject, the naltrexone is at least about 50% bioavailable. In certain embodiments, upon intranasal administration of the composition to a human subject, the naltrexone is at least about 70% bioavailable. In certain embodiments, upon storage of the composition at 40° C. and 75% relative humidity for a period of at least about 1 week, at least about 85%, of the original opioid receptor antagonist is still present in the composition.

Another aspect of the invention features an intranasally deliverable pharmaceutical composition, comprising an opioid receptor antagonist or salt thereof and a liquid carrier, said opioid receptor antagonist or salt thereof having a bioavailability of greater than about 30% when said pharmaceutical composition is administered to the nasal cavity of a human. In certain embodiments, the pharmaceutical composition comprises a therapeutically effective amount of an opioid receptor antagonist or salt thereof. In certain embodiments, the opioid receptor antagonist is naltrexone or salt thereof. In certain embodiments, the naltrexone or salt thereof is present in dissolved or solubilized form in the liquid carrier at a concentration of about 10 mg/mL to about 100 mg/mL.

In certain embodiments, the pharmaceutical composition further comprises at least one water miscible solvent. In certain embodiments, the pharmaceutical composition comprises about 0.5% (w/v) to about 5% (w/v) of a water miscible solvent. In certain embodiments, the water miscible solvent comprises propylene glycol, alcohol, glycerol, isopropyl alcohol or polyethylene glycol, or mixtures thereof. In certain embodiments, the water miscible solvent is glycerol. In certain embodiments, the water miscible solvent comprises methoxypolyethylene glycol, and the opioid receptor antagonist is naltrexone or salt thereof. In certain embodiments, the pharmaceutical composition further comprises a cellulose ether. In certain embodiments, the pharmaceutical composition comprises about 0.5% (w/v) to about 5% (w/v) of a cellulose ether. In certain embodiments, the cellulose ether is hydroxypropyl methylcellulose. In certain embodiments, the pharmaceutical composition further comprises a buffering agent. In certain embodiments, the pharmaceutical composition further comprises a preservative. In certain embodiments, the pharmaceutical composition has a pH in range of about 3.0 to about 6.5.

In certain embodiments, said opioid receptor antagonist or salt thereof has an absolute bioavailability of greater than about 50% when said pharmaceutical composition is administered to the nasal cavity of a human. In certain embodiments, said opioid receptor antagonist or salt thereof has an absolute bioavailability of greater than about 70% when said pharmaceutical composition is administered to the nasal cavity of a human. In certain embodiments, said opioid receptor antagonist or salt thereof has an absolute bioavailability in the range of about 50% to about 70% when said pharmaceutical composition is administered to the nasal cavity of a human.

### Exemplary Methods

One aspect of the invention features a method of treating and/or preventing alcohol cravings in a subject in need thereof, the method comprising intranasally administering to the subject a pharmaceutical composition comprising an opioid receptor antagonist or a salt thereof and a liquid carrier, wherein at least a portion of the opioid receptor antagonist or salt thereof is present in dissolved or solubilized form in the liquid carrier. In certain embodiments, the opioid receptor antagonist or salt thereof is naltrexone. In certain embodiments, the naltrexone or salt thereof is present in dissolved or solubilized form in the liquid carrier at a concentration of about 60 mg/mL to about 90 mg/mL.

In certain embodiments, the liquid carrier further comprises at least one water miscible solvent. In certain embodiments, the water miscible solvent comprises propylene glycol, alcohol, glycerol, isopropyl alcohol or polyethylene glycol, or mixtures thereof. In certain embodiments, upon intranasal administration of the composition to the subject, the naltrexone or salt thereof is at least about 50% bioavailable. In certain embodiments, the method relates to further comprising administering, in combination with the opioid receptor antagonist, one or more additional therapeutic agents selected from the group consisting of acamprosate, tiapride, ondansetron, sertraline, topirimate, aripiprazole and disulfiram. In certain embodiments, the opioid receptor antagonist or salt thereof has a relative bioavailability, as compared to an orally administered naltrexone dose, of greater than about 100% when the pharmaceutical composition is administered to the nasal cavity of a human.

Another aspect of the invention features a method for treating acute alcohol craving in a subject in need thereof, the method comprising intranasally administering to the subject a pharmaceutical composition comprising an opioid receptor antagonist or a salt thereof and a liquid carrier, wherein at least a portion of the opioid receptor antagonist or salt thereof is present in dissolved or solubilized form in the liquid carrier. In certain embodiments, the opioid receptor antagonist is naltrexone or a salt thereof. In certain embodiments, the naltrexone or salt thereof is present in dissolved or solubilized form in the liquid carrier at a concentration of about 10 mg/mL to about 100 mg/mL.

In certain embodiments, the liquid carrier further comprises at least one water miscible solvent. In certain embodiments, the water miscible solvent comprises propylene glycol, alcohol, glycerol, isopropyl alcohol or polyethylene glycol, or mixtures thereof. In certain embodiments, upon intranasal administration of the composition to the subject, the naltrexone or salt thereof is at least about 50% bioavailable. In certain embodiments, the opioid receptor antagonist or salt thereof has a relative bioavailability, as compared to an orally administered naltrexone dose, of greater than about 100% when the pharmaceutical composition is administered to the nasal cavity of a human.

Another aspect of the invention features a method for reducing a number of alcoholic drinks a subject consumes per day, the method comprising intranasally administering to the subject a pharmaceutical composition comprising an opioid receptor antagonist or a salt thereof and a liquid carrier, wherein at least a portion of the opioid receptor antagonist or salt thereof is present in dissolved or solubilized form in the liquid carrier. In certain embodiments, the opioid receptor antagonist is naltrexone or a salt thereof. In certain embodiments, the naltrexone or salt thereof is present in dissolved or solubilized form in the liquid carrier at a concentration of about 10 mg/mL to about 100 mg/mL.

In certain embodiments, the liquid carrier further comprises at least one water miscible solvent. In certain embodiments, the water miscible solvent comprises propylene glycol, alcohol, glycerol, isopropyl alcohol or polyethylene glycol, or mixtures thereof. In certain embodiments, upon intranasal administration of the composition to the subject, the naltrexone or salt thereof is at least about 50% bioavailable. In certain embodiments, the opioid receptor antagonist or salt thereof has a relative bioavailability, as compared to an orally administered naltrexone dose, of greater than about 100% when the pharmaceutical composition is administered to the nasal cavity of a human.

Another aspect of the invention features a method of treating or preventing alcoholism in a human, comprising administering intranasally to a human in need thereof a therapeutically effective amount of a pharmaceutical composition comprising an opioid receptor antagonist or a salt thereof and a liquid carrier, wherein said opioid receptor antagonist or salt thereof has an absolute bioavailability of greater than about 30% when said pharmaceutical composition is administered to the nasal cavity of a human. In certain embodiments, the opioid receptor antagonist is naltrexone or salt thereof. In certain embodiments, the naltrexone or salt thereof is present in dissolved or solubilized form in the liquid carrier at a concentration of about 10 mg/mL to about 100 mg/mL.

In certain embodiments, the pharmaceutical composition further comprises at least one water miscible solvent. In certain embodiments, the pharmaceutical composition comprises about 0.5% w/v to about 5% w/v of a water miscible solvent. In certain embodiments, the water miscible solvent comprises propylene glycol, alcohol, glycerol, isopropyl alcohol or polyethylene glycol, or mixtures thereof. In certain embodiments, the water miscible solvent is glycerol. In certain embodiments, the water miscible solvent comprises methoxypolyethylene glycol, and the opioid receptor antagonist is naltrexone or salt thereof. In certain embodiments, the pharmaceutical composition further comprises a cellulose ether. In certain embodiments, the pharmaceutical composition comprises about 0.5% (w/v) to about 5% (w/v) of a cellulose ether. In certain embodiments, the cellulose ether is hydroxypropyl methylcellulose. In certain embodiments, the pharmaceutical composition further comprises a buffering agent. In certain embodiments, the pharmaceutical composition further comprises a preservative. In certain embodiments, the pharmaceutical composition has a pH in range of about 3.0 to about 6.5.

In certain embodiments, said opioid receptor antagonist or salt thereof has an absolute bioavailability of greater than about 50% when said pharmaceutical composition is administered to the nasal cavity of a human. In certain embodiments, said opioid receptor antagonist or salt thereof has an absolute bioavailability of greater than about 70% when said pharmaceutical composition is administered to the nasal cavity of a human. In certain embodiments, said opioid receptor antagonist or salt thereof has an absolute bioavailability in the range of about 50% to about 70% when said pharmaceutical composition is administered to the nasal cavity of a human. In certain embodiments, a single intranasal administration of said pharmaceutical composition is sufficient to deliver a dose in the range of about 2 mg to about 8 mg of said opioid receptor antagonist to the nasal cavity of said human. In certain embodiments, upon intranasal administration of a composition of the invention to a human subject, the subject exhibits one or more of: a Tₘₐₓ naltrexone plasma concentration of less than about 0.75 hr; a Cₘₐₓ naltrexone plasma concentration of at least about 2 ng/mL; and/or an AUC naltrexone plasma concentration of at least about 5 ng*hr/mL.

In certain embodiments, the method further comprises administering one or more additional therapeutic agents selected from the group consisting of acamprosate, tiapride, ondansetron, sertraline, topirimate, aripiprazole and disulfiram. In certain embodiments, the opioid receptor antagonist or salt thereof has a relative bioavailability, as compared to an orally administered naltrexone dose, of greater than about 100% when the pharmaceutical composition is administered to the nasal cavity of a human.

Another aspect of the invention features a method of treating acute alcohol craving in a human, comprising administering intranasally to a human in need thereof a therapeutically effective amount of a pharmaceutical composition comprising an opioid receptor antagonist or a salt thereof and a liquid carrier, wherein at least a portion of the opioid receptor antagonist or salt thereof is present in dissolved or solubilized form in the liquid carrier. In certain embodiments, the opioid receptor antagonist is naltrexone or a salt thereof. In certain embodiments, the naltrexone or salt thereof is present in dissolved or solubilized form in the liquid carrier at a concentration of about 10 mg/mL to about 100 mg/mL.

In certain embodiments, the pharmaceutical composition further comprises at least one water miscible solvent. In certain embodiments, the pharmaceutical composition comprises about 0.5% (w/v) to about 5% (w/v) of a water miscible solvent. In certain embodiments, the water miscible solvent comprises propylene glycol, alcohol, glycerol, isopropyl alcohol or polyethylene glycol, or mixtures thereof. In certain embodiments, the water miscible solvent is glycerol. In certain embodiments, the water miscible solvent comprises methoxypolyethylene glycol, and the opioid receptor antagonist is naltrexone or salt thereof. In certain embodiments, the pharmaceutical composition further comprises a cellulose ether. In certain embodiments, the pharmaceutical composition comprises about 0.5 wt% to about 5 wt% of a cellulose ether. In certain embodiments, the cellulose ether is hydroxypropyl methylcellulose. In certain embodiments, the pharmaceutical composition further comprises a buffering agent. In certain embodiments, the pharmaceutical composition further comprises a preservative. In certain embodiments, the pharmaceutical composition has a pH in range of about 3.0 to about 6.5.

In certain embodiments, said opioid receptor antagonist or salt thereof has a bioavailability of greater than about 50% when said pharmaceutical composition is administered to the nasal cavity of a human. In certain embodiments, said opioid receptor antagonist or salt thereof has a bioavailability of greater than about 70% when said pharmaceutical composition is administered to the nasal cavity of a human. In certain embodiments, said opioid receptor antagonist or salt thereof has a bioavailability in the range of about 50% to about 70% when said pharmaceutical composition is administered to the nasal cavity of a human. In certain embodiments, a single intranasal administration of said pharmaceutical composition is sufficient to deliver a dose in the range of about 2 mg to about 8 mg of said opioid receptor antagonist to the nasal cavity of said human. In certain embodiments, upon intranasal administration of a composition of the invention to a human subject, the subject exhibits one or more of: a Tₘₐₓ naltrexone plasma concentration of less than about 0.75 hr; a Cₘₐₓ naltrexone plasma concentration of at least about 2 ng/mL; and/or an AUC naltrexone plasma concentration of at least about 5 ng*hr/mL.

In certain embodiments, the method further comprises administering one or more additional therapeutic agents selected from the group consisting of acamprosate, tiapride, ondansetron, sertraline, topirimate, aripiprazole and disulfiram. In certain embodiments, the opioid receptor antagonist or salt thereof has a relative bioavailability, as compared to an orally administered naltrexone dose, of greater than about 100% when the pharmaceutical composition is administered to the nasal cavity of a human.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

The aqueous formulations listed in Table 1 were prepared for intranasal administration.

TABLE 1

| Formulation | Ingredients | Physical Properties |
|---|---|---|
| A | 25 mg/mL naltrexone | pH = 4.3 |
| | 25 mM potassium phosphate | osmo = 348 |
| | 1.5% glycerin | |
| | 0.5% sodium benzoate | |
| B | 50 mg/mL naltrexone | pH = 6.1 |
| | 0.9% NaCl | osmo = 467 |
| C | 50 mg/mL naltrexone | pH = 3.125 |
| | 2.5% hydroxypropyl methylcellulose | osmo = 237 |
| | WFI water | |
| D | 50 mg/mL naltrexone | pH = 4.559 |
| | 2.5% mPEG | osmo = 763 mOsm |
| | 2.5% glycerin | |
| | WFI water | |
| E | 50 mg/mL naltrexone | osmo = 204 mOsm |
| | 1.0% hypromellose | |
| | WFI water | |

### Example 2

It is contemplated that the above formulations may be administered intranasally to a patient suffering from alcoholism. The dosage and dosing schedule can be determined by one of ordinary skill in the art using standard procedures.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respect illustrative rather than limiting the invention described herein. Scope of the invention is thus indicated by the appended claims rather than by the foregoing description, and all changes that come with in the meaning and range of equivalency of the claims are intended to be embraced therein.

What is claimed is:

1. An intranasally deliverable pharmaceutical composition, comprising an opioid receptor antagonist or salt thereof and a liquid carrier, said opioid receptor antagonist or salt thereof having an absolute bioavailability of greater than about 30% when said pharmaceutical composition is administered to the nasal cavity of a human.

2. The pharmaceutical composition of claim 1, wherein the opioid receptor antagonist is naltrexone or salt thereof.

3. The pharmaceutical composition of claim 2, wherein the naltrexone or salt thereof is present in dissolved or solubilized form in the liquid carrier at a concentration of about 10 mg/mL to about 100 mg/mL.

4. The pharmaceutical composition of claim 1, wherein the pharmaceutical composition further comprises at least one water miscible solvent.

5. The pharmaceutical composition of claim 4, wherein the water miscible solvent comprises propylene glycol, alcohol, glycerol, isopropyl alcohol, polyethylene glycol, or mixtures thereof.

6. The pharmaceutical composition of claim 1, wherein the pharmaceutical composition further comprises a cellulose ether.

7. The pharmaceutical composition of claim 6, wherein the cellulose ether is hydroxypropyl methylcellulose.

8. The pharmaceutical composition of claim 1, wherein said opioid receptor antagonist or salt thereof has an absolute bioavailability of greater than about 50% when said pharmaceutical composition is administered to the nasal cavity of a human.

9. A method of treating or preventing alcoholism in a human, comprising administering intranasally to a human in need thereof a therapeutically effective amount of a pharmaceutical composition comprising an opioid receptor antagonist or a salt thereof and a liquid carrier, wherein said opioid receptor antagonist or salt thereof has an absolute bioavailability of greater than about 30% when said pharmaceutical composition is administered to the nasal cavity of a human.

10. The method of claim 9, wherein the opioid receptor antagonist is naltrexone or salt thereof.

11. The method of claim 10, wherein the naltrexone or salt thereof is present in dissolved or solubilized form in the liquid carrier at a concentration of about 10 mg/mL to about 100 mg/mL.

12. The method of claim 9, wherein the pharmaceutical composition further comprises at least one water miscible solvent.

13. The method of claim 12, wherein the water miscible solvent comprises propylene glycol, alcohol, glycerol, isopropyl alcohol, polyethylene glycol, or mixtures thereof.

14. The method of claim 9, wherein the pharmaceutical composition further comprises a cellulose ether.

15. The method of claim 14, wherein the cellulose ether is hydroxypropyl methylcellulose.

16. The method of claim 9, wherein the pharmaceutical composition has a pH in range of about 3.0 to about 6.5.

17. The method of claim 9, wherein said opioid receptor antagonist or salt thereof has an absolute bioavailability of greater than about 50% when said pharmaceutical composition is administered to the nasal cavity of a human.

18. The method of claim 9, wherein a single intranasal administration of said pharmaceutical composition is sufficient to deliver a dose in the range of about 2 mg to about 8 mg of said opioid receptor antagonist to the nasal cavity of said human.

19. The method of claim 10, wherein upon intranasal administration of said composition to a human subject, the subject exhibits one or more of: a Tₘₐₓ naltrexone plasma concentration of less than about 0.75 hr; a Cₘₐₓ naltrexone plasma concentration of at least about 2 ng/mL; and/or an AUC naltrexone plasma concentration of at least about 5 ng*hr/mL.

20. A method of treating acute alcohol craving in a human, comprising administering intranasally to a human in need thereof a therapeutically effective amount of a pharmaceutical composition comprising an opioid receptor antagonist or a salt thereof and a liquid carrier, wherein at least a portion of the opioid receptor antagonist or salt thereof is present in dissolved or solubilized form in the liquid carrier.
